(19) United States
(12) Patent Application Publication
(10) Pub. No.: US 2026/0358523 A1
(43) Pub. Date: Nov. 12, 2026

(54) FOUR-DIMENSIONAL (4D) ULTRASOUND IMAGING SYSTEM AND METHOD BASED ON ELECTRODE SEGMENTATION AND COMPRESSIVE SENSING

(71) Applicant: ZHEJIANG UNIVERSITY, Hangzhou (CN)

(72) Inventors: Jian CHEN, Hangzhou (CN); Youlong HUA, Hangzhou (CN); Mengru ZHANG, Hangzhou (CN); Zhenyang YU, Hangzhou (CN)

(73) Assignee: ZHEJIANG UNIVERSITY, Hangzhou (CN)

(21) Appl. No.: 19/450,820

(22) Filed: Jan. 16, 2026

(30) Foreign Application Priority Data
May 6, 2025 (CN) 202510571564.9

(51) Int. Cl. G01V 1/30; A61B 8/00; B06B 1/06; G01V 1/18

(52) U.S. Cl. CPC G01V 1/308; B06B 1/0629; G01V 1/186; A61B 8/48

(57) ABSTRACT

A four-dimensional (4D) ultrasound imaging system based on electrode segmentation and compressive sensing includes a backing, a piezoelectric ceramic, and a matching layer that are sequentially disposed in a housing; a positive electrode surface of the piezoelectric ceramic serves as an ultrasound radiation surface and faces a target region through the matching layer; the backing configured to absorb a reflected wave is disposed on a negative electrode surface of the piezoelectric ceramic; the piezoelectric ceramic is electrically connected to a control circuit via a cable; a spatial phase mask is disposed outside the housing, comes into contact with a side of the matching layer away from the piezoelectric ceramic, and is configured to modulate a spatial phase distribution of a transmitted ultrasound wave; the ultrasound radiation surface of the piezoelectric ceramic is segmented into multiple electrode regions; the electrode regions are electrically connected to the control circuit.

### CROSS REFERENCE TO THE RELATED APPLICATIONS

This application is based upon and claims priority to Chinese Patent Application No. 202510571564.9, filed on May 6, 2025, the entire contents of which are incorporated herein by reference.

### TECHNICAL FIELD

The present disclosure relates to the technical field of ultrasonic non-destructive testing, and in particular to a four-dimensional (4D) ultrasound imaging system and method based on electrode segmentation and compressive sensing.

### BACKGROUND

Ultrasound imaging is widely applied to industrial testing and biomedicine. By moving an ultrasound probe, images of a tested cross-section can be obtained. The ultrasound probe can transmit an ultrasound pulse signal (typically at a frequency of 1-20 MHz), while receiving a reflected echo from the target region. The position of an imaged object can be calculated by analyzing the time between the transmitted ultrasound pulse signal and the reflected echo. The common phased-array ultrasound probe is mainly made of piezoelectric ceramic arranged in an array. When a voltage is applied to the material, the piezoelectric crystal vibrates and produces a short ultrasound pulse of a few cycles. When relevant array elements are excited with appropriate delays, the transmitted ultrasound wave propagates in the form of a narrow beam and can be focused on a point of interest. These focused beams can be used to scan a whole volume. When the signal is received, similar delays can be used to selectively enhance the signals from scatterers along the beam of interest. This type of focusing that uses delayed signals in transmission and/or reception is named beamforming and has been widely researched and put into use for many years.

As an interdisciplinary integrated innovative achievement, 4D imaging (real-time three-dimensional (3D) dynamic imaging) has become a relatively cutting-edge research direction. In the field of industrial non-destructive testing, 4D ultrasound has been applied to the real-time 3D localization of defects inside materials. In the field of biomedicine, 4D ultrasound combined with Doppler hemodynamic analysis makes it possible to perform the kinematic analysis of cardiac valves and the quantitative evaluation of tumor angiogenesis. However, existing real-time 3D dynamic imaging relies on the optimization of beamforming for the phased-array ultrasound probe and the iteration of parallel computing architectures. After analog-to-digital conversion, the individual signal received by each array element needs to be post-processed by dozens of array elements, complex hardware, and cumbersome signal processing logic. Very recently, the discovery of compressive sensing has shown that the signal structure can be exploited to reduce the burden posed by requirements of the conventional phased-array ultrasound imaging.

Compressive sensing is mainly intended to break the phase uniformity of ultrasound waves during the transmission and reception processes, thereby projecting 3D object information onto a set of incoherent basis functions. Specifically, a phase encoding mask is placed in front of the ultrasound probe to change propagation time of the ultrasound waves to the target medium. At this time, the phase uniformity of the ultrasound waves is broken, and complex spatiotemporal interference patterns are generated. Consequently, each position (each pixel) in the 3D space produces a unique ultrasound signal from its position. Echo signals of all pixels are acquired by the ultrasound probe through the mask to obtain compressed measurements. At this time, the real scene signal can be reconstructed by finding inverse solutions to the compressed measurements. Because of the limitations in temporal bandwidth, as well as in mask specific phase distribution, it is not guaranteed that the echo signals of all pixels are uncorrelated. In view of this, researchers rotate or move the mask to obtain additional signal measurements, but this is difficult to operate and implement in actual testing.

### SUMMARY

In order to solve the above technical problem, the present disclosure provides a 4D ultrasound imaging system and method based on electrode segmentation and compressive sensing.

The present disclosure adopts the following technical solutions:
A 4D ultrasound imaging system based on electrode segmentation and compressive sensing includes a housing, a cable, a backing, a piezoelectric ceramic, a matching layer, a spatial phase mask, and a control circuit, where the backing, the piezoelectric ceramic, and the matching layer are sequentially disposed in the housing; a positive electrode surface of the piezoelectric ceramic serves as an ultrasound radiation surface and faces a target region; the backing configured to absorb a reflected wave is disposed on a negative electrode surface of the piezoelectric ceramic; the piezoelectric ceramic is electrically connected to the control circuit via the cable extending out of the housing; and the spatial phase mask is disposed outside the housing, comes into contact with a side of the matching layer away from the piezoelectric ceramic, and is configured to modulate a spatial phase distribution of a transmitted ultrasound wave; and
the ultrasound radiation surface of the piezoelectric ceramic is segmented into L×L electrode regions; each of the electrode regions is electrically connected to the control circuit; the control circuit is configured to control independent excitation and reception of each of the electrode regions; and a negative electrode of the piezoelectric ceramic is grounded.

The 4D ultrasound imaging system based on electrode segmentation and compressive sensing further includes a linear compressive sensing module, where the linear compressive sensing module is electrically connected to the control circuit, and configured to reconstruct a 4D ultrasound image of the target region; and
a locking knob, where the locking knob is disposed on a top surface of the housing, and configured to fix the cable extending out of the housing.

A size of the spatial phase mask is the same as a size of the piezoelectric ceramic; the spatial phase mask is segmented into L×L mask regions; the L×L mask regions have different thicknesses; a thickness relation between the L×L mask regions is configured such that a spatial phase difference between transmitted waves of any two of the L×L mask regions in the spatial phase mask is greater than or equal to 0, and less than or equal to π; and the spatial phase difference between the transmitted waves of the two mask regions is set by:

φ=arg(P1−P2)
where φ represents the spatial phase difference between the transmitted waves, arg( ) represents a complex argument, and P1 and P2 are respectively complex representations of the transmitted waves of the two mask regions.

Both the backing and the matching layer are made of epoxy resin mixed with tungsten powder; a mass ratio of the tungsten powder to the epoxy resin in the backing is (2-4):1; a mass ratio of the tungsten powder to the epoxy resin in the matching layer is 1:(3-5); and a thickness of the matching layer is a quarter of a wavelength at a center frequency of the piezoelectric ceramic.

The ultrasound radiation surface of the piezoelectric ceramic is segmented into a plurality of electrode regions in any one of following methods: laser cutting, dicing, or sputtering deposition of a metal electrode layer on the piezoelectric ceramic.

The ultrasound radiation surface of the piezoelectric ceramic is provided with a multi-electrode flexible printed circuit (FPC) board or pin electrodes.

A 4D ultrasound imaging method includes following steps:
S1: performing wave field scanning on the target region to obtain a system matrix, and establishing a linear system model according to the system matrix;
S2: randomly exciting an electrode region of the piezoelectric ceramic to emit an ultrasound wave, where the ultrasound wave is modulated by the spatial phase mask and then reflected by the target region to obtain an echo signal;
S3: allowing the echo signal to pass through the spatial phase mask and to be received by the piezoelectric ceramic to form a compressed signal, and recording, by the control circuit, the compressed signal and transmitting the compressed signal to the linear system model; and
S4: repeating the step S2 and the step S3 sequentially, with the electrode regions of the piezoelectric ceramic being randomly selected for excitation each time until the linear system model receives S compressed signals, and reconstructing, by the linear system model, the 4D ultrasound image of the target region according to the S compressed signals.

The wave field scanning is performed as follows:
acquiring, by using a hydrophone in a plane perpendicular to an ultrasound propagation direction, the ultrasound wave modulated by the spatial phase mask, processing the ultrasound wave modulated by the spatial phase mask to obtain wave field data, propagating the wave field data with an angular spectrum method to planes at different propagation distances to obtain wave field data at the different propagation distances, and constructing the system matrix according to the wave field data at the different propagation distances.

The linear system model reconstructs the 4D ultrasound image of the target region by:

u=Hv+n
where u represents the compressed signal, v represents a reconstructed 4D ultrasound image signal, n represents a noise signal, and H represents the system matrix.

The present disclosure has the following beneficial effects:

The present disclosure realizes the 3D dynamic imaging in combination with the single electrode-segmented ultrasound transducer and the phase mask. Compared with complex and expensive phased array systems and other signal acquisition methods, the present disclosure significantly reduces the cost, and greatly enhances the operational portability of the device. By randomly exciting different regions each time to acquire multiple sets of “multiple-transmit single-receive” signals, the present disclosure effectively solves the prior art problem of difficulty in rotating or moving the mask to obtain additional signal measurements.

### BRIEF DESCRIPTION OF THE DRAWINGS

FIG. 1 is a schematic assembly view of a 4D ultrasound imaging system;

FIG. 2 is an exploded view of a 4D ultrasound imaging system;

FIG. 3 is a schematic view of electrode wiring of a 4D ultrasound imaging system;

FIG. 4 is a schematic structural view of a spatial phase mask of a 4D ultrasound imaging system;

FIG. 5 is a schematic view of a linear compressive sensing module of a 4D ultrasound imaging system;

FIG. 6 is a flowchart of a 4D ultrasound imaging method;

FIG. 7 is a schematic view of a simulated defect in Embodiment 1 of a 4D ultrasound imaging method;

FIG. 8 illustrates an imaging result in Embodiment 1 of a 4D ultrasound imaging method;

FIG. 9 is a schematic view of a simulated defect in Embodiment 2 of a 4D ultrasound imaging method; and

FIG. 10 illustrates an imaging result in Embodiment 2 of a 4D ultrasound imaging method.

### REFERENCE NUMERALS

1: housing, 2: locking knob, 3: cable, 4: backing, 5: piezoelectric ceramic, 6: matching layer, and 7: spatial phase mask.

### DETAILED DESCRIPTION OF THE EMBODIMENTS

The present disclosure will be further described below in conjunction with description of drawings and embodiments, but the implementation of the present disclosure includes but is not limited to the following embodiments.

It should be understood that the directional or positional relations indicated by the terms “hollow”, “top”, “bottom”, “inside”, “outside”, and the like are only used to facilitate and simplify the description of the present disclosure, and rather than to indicate or imply that components or elements referred to must have specific directions or must be formed and operated in specific directions. Therefore, these terms should not to be understood as a limitation to the present disclosure.

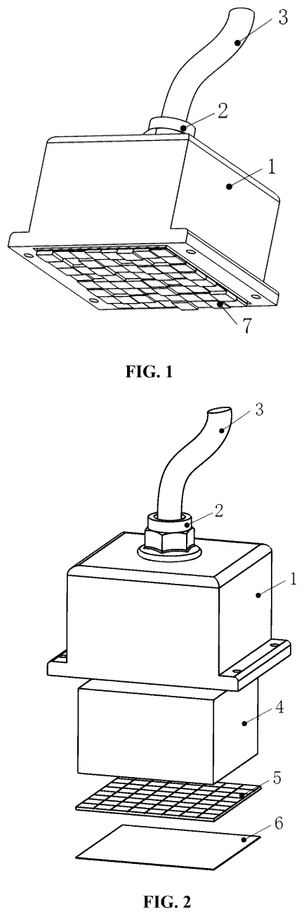

As shown in FIG. 1 and FIG. 2, a 4D ultrasound imaging system includes housing 1, cable 3, backing 4, piezoelectric ceramic 5, matching layer 6, spatial phase mask 7, and a control circuit. The backing 4, the piezoelectric ceramic 5, and the matching layer 6 are sequentially disposed in the housing 1. A positive electrode surface of the piezoelectric ceramic 5 serves as an ultrasound radiation surface and is configured to emit an ultrasound wave to a target region. The backing 4 configured to absorb a reflected wave is disposed on a negative electrode surface of the piezoelectric ceramic 5. The piezoelectric ceramic 5 is electrically connected to the control circuit via the cable 3 extending out of the housing 1. The spatial phase mask 7 is disposed outside the housing 1, comes into contact with a side of the matching layer 6 away from the piezoelectric ceramic 5, and is configured to modulate a spatial phase distribution of a transmitted ultrasound wave.

The ultrasound radiation surface of the piezoelectric ceramic 5 is segmented into L×L electrode regions. Each electrode region is electrically connected to the control circuit. The control circuit is configured to control independent excitation and ultrasound wave reception of each electrode region, so the control circuit can excite a single electrode region or multiple electrode regions. A negative electrode of the piezoelectric ceramic 5 is grounded.

The reflected wave absorbed by the backing 4 is produced when the ultrasound wave emitted from the piezoelectric ceramic 5 impinges on the backing 4. The backing 4 is configured to prevent the reflected wave from coming back to the piezoelectric ceramic 5.

Two sides of the piezoelectric ceramic 5 are respectively provided with electrode layers. The electrode layers are electrically connected to a driving output terminal of the control circuit through the cable 3. The control circuit may be connected to an oscilloscope, through which a compressed signal can be observed and analyzed.

Unlike phased array ultrasound transducers, the ultrasound radiation surface of the piezoelectric ceramic 5 and the control circuit thereof neither involve complex logic circuits, nor control the excitation time sequence of each array element through an electronic delay. The control circuit realizes multi-element cooperative control such as single-transmit-all-receive and multiple-transmit-all-receive on the electrode regions, so the structure is simpler than the existing 4D ultrasound imaging system.

The cable 3 includes a plurality of wires. Each electrode region is connected to the control circuit via one wire. Since the negative electrode of the piezoelectric ceramic 5 is connected to a ground terminal of the control circuit via one wire, the cable 3 includes one more wire than the number of electrode regions.

The 4D ultrasound imaging system further includes a linear compressive sensing module. The linear compressive sensing module is electrically connected to the control circuit, and configured to receive the compressed signal transmitted from the control circuit, thereby reconstructing a 4D ultrasound image of the target region.

The 4D ultrasound imaging system further includes locking knob 2. The locking knob 2 is disposed on a top surface of the housing 1, and configured to fix the cable 3 extending out of the housing 1.

The spatial phase mask is segmented according to the number of electrode regions. In one region of the mask, a modulated transmitted ultrasound field for an incident plane wave with a frequency of f can be expressed as P=e^{i(k}^{ₐ}^{h)}, where vₐ is a sound velocity of the mask material, and kₐ is a wave velocity of the mask material. kₐ=2πf/vₐ is the wave number of the material. Corresponding to different thicknesses h, the transmitted ultrasound field is different.

As shown in FIG. 4, a size of the spatial phase mask 7 is the same as a size of the piezoelectric ceramic 5. The spatial phase mask 7 is segmented into L×L mask regions in the same segmentation method as the electrode regions on the ultrasound radiation surface of the piezoelectric ceramic 5. The L×L mask regions have different thicknesses. A thickness relation between the L×L mask regions is configured such that a spatial phase difference between transmitted waves of any two of the L×L mask regions in the spatial phase mask 7 is greater than or equal to 0, and less than or equal to π. The spatial phase difference between the transmitted waves is set by:

φ=arg(P1−P2)
where φ represents the spatial phase difference between the transmitted waves, arg( ) represents a complex argument, and P1 and P2 are respectively complex representations of the transmitted waves of the two mask regions.

z=r(cos θ+i sin θ), where r is a modulus of z, r=|z|, θ is a complex argument of z, and θ=arg(z).

The transmitted waves are waves modulated by the spatial phase mask 7. The spatial phase difference between the transmitted waves is a phase difference between two transmitted waves.

The spatial phase mask 7 can be directly assembled in front of a single probe with segmented electrodes after being fabricated. Spatial phases of the mask can be randomly distributed. The spatial phase mask 7 is fabricated using a method including but not limited to subtractive manufacturing on a flat plate, 3D printing or use of a phase-change polymer material with relevant changes on the sound velocity. At this time, the phase uniformity of the transmitted wave is broken, thereby forming a random deterministic interference pattern in the medium. Generally, additive manufacturing such as 3D printing or subtractive manufacturing such as regional drilling can be selected to fabricate the spatial phase mask with random thickness regions. It is to be noted that the selected material must have a small acoustic attenuation coefficient and an acoustic impedance relatively matching water, so as to ensure the desirable ultrasound wave transmission efficiency.

Specifically, the spatial phase mask 7 is segmented into a plurality of mask regions in the same segmentation method as the electrode regions on the ultrasound radiation surface of the piezoelectric ceramic 5. That is, the mask regions segmented by the spatial phase mask 7 are the same as the electrode regions segmented by the ultrasound radiation surface of the piezoelectric ceramic 5 in quantity and size. The mask regions segmented by the spatial phase mask 7 are in one-to-one correspondence with the electrode regions segmented by the ultrasound radiation surface of the piezoelectric ceramic 5.

Both the backing 4 and the matching layer 6 are made of epoxy resin mixed with tungsten powder. A mass ratio of the tungsten powder to the epoxy resin in the backing 4 is (2-4):1. A mass ratio of the tungsten powder to the epoxy resin in the matching layer 6 is 1:(3-5). A thickness of the matching layer 6 is a quarter of a wavelength at a center frequency of the piezoelectric ceramic 5.

The matching layer is uniformly spin-coated. Generally, the thickness of the matching layer is preferably a quarter of a wavelength at a center frequency of a piezoelectric wafer in water.

The ultrasound radiation surface of the piezoelectric ceramic 5 is segmented into a plurality of electrode regions in any one of following methods: laser cutting, dicing, or sputtering deposition of a metal electrode layer on the piezoelectric ceramic 5, i.e., addition of segmented masks during sputtering of the original metal electrode coating.

Electrodes on one side of the piezoelectric ceramic 5 are segmented with laser or other dicing devices. Segmented electrodes each are connected to a separate wire. Electrodes on the other side of the wafer may share one wire. Generally, the piezoelectric ceramic 5 preferably includes the positive electrode subjected to electrode segmentation, and the negative electrode connected to a ground circuit. It is to be noted that while the electrodes on the surface of the piezoelectric ceramic 5 are segmented, a segmentation depth may be provided, so as to ensure each single region may be independently excited.

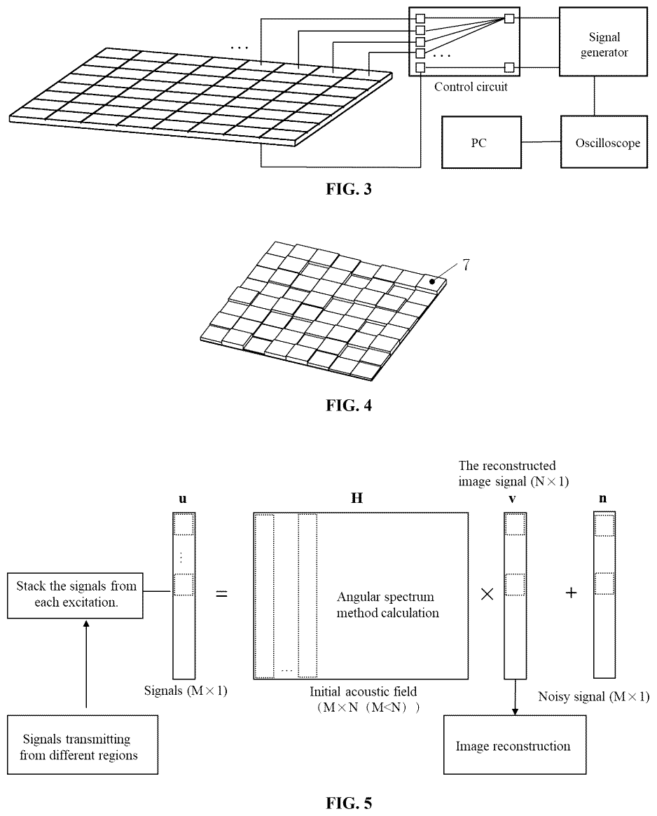

FIG. 3 is a schematic view of a control circuit corresponding to the single probe with the segmented electrodes. Taking a single-probe transducer with 64 electrode regions as an example, a positive lead in each region of the probe is independently connected to an analog electronic switch on the controller, while a negative lead is grounded. The analog electronic switch of the controller can independently control on-off of the signal at a high speed. Further, a single region or multiple regions of the single probe can be excited, while a Bayonet Neill-Concelman (BNC) interface connected to a signal generator is reserved to receive and transmit an echo signal.

The ultrasound radiation surface of the piezoelectric ceramic 5 is provided with a multi-electrode FPC board or pin electrodes. The negative electrode of the piezoelectric ceramic 5 is welded with a ground wire and grounded.

The 4D ultrasound imaging system follows the concept central to compressive imaging: The object/image information is projected through a set of incoherent functions onto single measurements, and the retrieved measurements are then decoded to form an image. In view of this, the single probe with the segmented electrodes is provided. By randomly modulating the emitted ultrasound wave with the mask, the phase uniformity of the wave field is broken. The phase delay mask can ensure that each region in the image is uniquely identifiable in the compressed measurement. By randomly exciting different regions each time, multiple sets of “multiple-transmit single-receive” signals are acquired. With real-time post-processing on the signals, the 3D dynamic imaging can be realized.

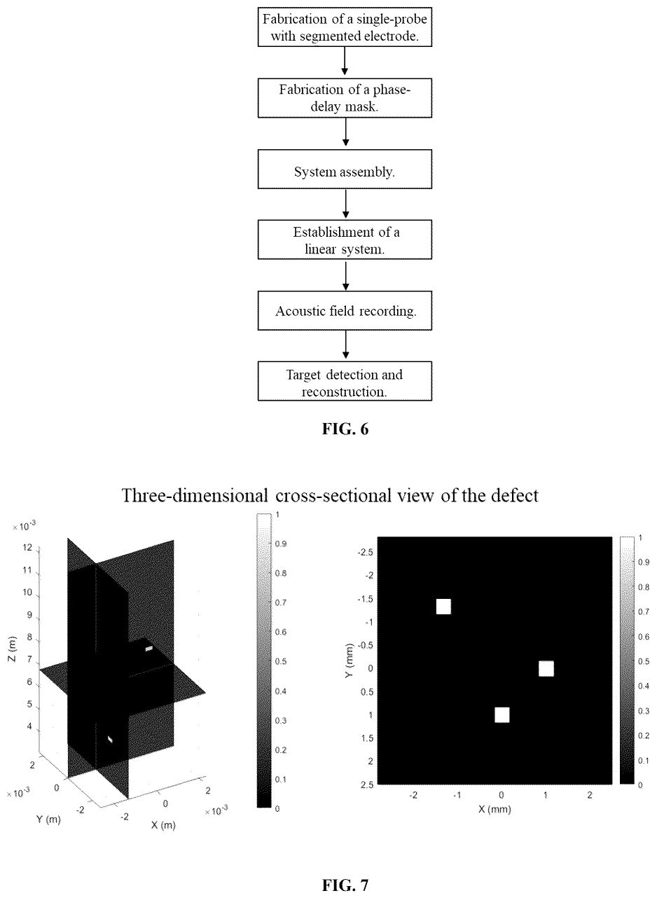

As shown in FIG. 6, a 4D ultrasound imaging method includes following steps:

S1: Wave field scanning is performed on the target region with a hydrophone to obtain system matrix H, and a linear system model is established according to the system matrix H.

The 4D ultrasound imaging system can be applied to fields such as underwater ultrasound testing and medical ultrasound. The target region refers to a spatial region in need of 4D imaging, and may be underwater testing objects or human tissues or the like.

Since the acoustic impedance of the human body is close to that of water, the system matrix obtained in water can still be used in medical ultrasound applications.

S2: An electrode region of the piezoelectric ceramic 5 is randomly excited to emit the ultrasound wave. The ultrasound wave is modulated by the spatial phase mask 7 and then reflected by the target region to obtain an echo signal.

S3: Due to phase modulation of the mask, reflected signals from different spatial positions are superimposed in an incoherent manner. The echo signal passes through the spatial phase mask 7 and is received by the piezoelectric ceramic 5 to form a compressed signal. The compressed signal is recorded by the control circuit and transmitted to the linear system model.

S4: The step S2 and the step S3 are repeated sequentially, with the electrode regions of the piezoelectric ceramic 5 being randomly selected for excitation each time until the linear system model receives S compressed signals, and the linear system model obtains enough information for reconstruction. The linear system model reconstructs the 4D ultrasound image of the target region according to the S compressed signals.

The wave field scanning is performed as follows:

The ultrasound wave modulated by the spatial phase mask 7 is acquired by using a small hydrophone in a plane close to a surface of the spatial phase mask 7 and perpendicular to an ultrasound propagation direction. The ultrasound wave modulated by the spatial phase mask 7 is processed to obtain wave field data. The wave field data is propagated with an angular spectrum method to planes at different propagation distances to obtain wave field data at the different propagation distances. The system matrix is constructed according to the wave field data at the different propagation distances.

The plane during the wave field scanning is perpendicular to the ultrasound propagation direction.

The block matrix H needs to be acquired in advance before the imaging, so as to know the whole spatiotemporal wave field. Further, the small hydrophone may be used to acquire a pulse signal in the plane close to the surface of the mask and an ultrasound transmission axis. The recorded wave field is then transmitted with the angular spectrum method to any plane parallel to the recorded plane.

Specifically, the transmitted ultrasound field acquired by the hydrophone is as follows:

p₀(x,y,z=0)=e^{(iφ0)}
where p₀(x, y, z=0) represents an initial distribution of the transmitted ultrasound field on plane z=0, and represents a state of the ultrasound field on the plane z=0.

A frequency spectrum of the plane at this time may be calculated by Fourier transform:

P₀(kₓ,k_{y},z=0)=∫∫p₀(x,y,z=0)dxdy
where P₀(kₓ, k_{y}, z=0) is the frequency spectrum of the transmitted ultrasound field, and represents a frequency domain on the plane z=0, and kₓ and k_{y} are respectively spatial wave numbers of the frequency spectrum in x and y directions.

Then, the transmitted ultrasound field is transmitted in a positive direction of the z-axis, and the frequency spectrum is multiplied by a phase shift factor:

H(kₓ,k_{y},z=1)=e^{(ikzl)}

Then, a frequency spectrum of any plane z=1 may be obtained:

P′(kₓ,k_{y},z=1)=P₀(kₓ,k_{y},z=0)*H(kₓ,k_{y},z=1)

An ultrasound field distribution on the plane may be obtained at this time by Fourier transform:

p′(x,y,z=1)=∫∫P′(kₓ,k_{y},z=1)dkₓdk_{y}
where p₀ is an initial ultrasound field acquired by the hydrophone when z=0, φ₀ is a phase of the initial ultrasound field acquired by the hydrophone when z=0, and H is the phase shift factor, and is used to adjust a transmitted frequency spectrum.

According to the reciprocity theorem, the pulse-echo signal is obtained by performing an autoconvolution of the transmitted hydrophone signal. Hence, the pulse-echo ultrasound signal at each point in the 3D space can be calculated. After repeated random switching, H columns can be filled by means of acquisition and calculation.

The linear system model reconstructs the 4D ultrasound image of the target region by:

u=Hv+n
where u represents the compressed signal, i.e., an actual testing signal, v represents a reconstructed 4D ultrasound image signal, i.e., an unknown real image, n represents a noise signal, and H represents the system matrix, i.e., a pulse-echo ultrasound signal at each point in an original spatiotemporal wave field.

Specifically, the system matrix H in the linear system model needs to be acquired in advance. After the linear system model is established, image reconstruction can be completed by inversely solving the vector v according to the compressed signal u. Randomly exciting different regions each time acquires multiple sets of “multiple-transmit full-receive” signals. With repeated random excitation, the ambiguity of the reconstructed image can be eliminated, and the signal-to-noise ratio (SNR) of the tested object can be greatly improved, thereby realizing efficient 3D reconstruction on the tested object. Based on fast switching characteristics for a plurality of electrode regions on the ultrasound radiation surface, random acquisitions and processing can be performed repeatedly and consecutively within a certain period of time. In combination with movement and acquisition of the probe, the 3D dynamic imaging can be realized.

Unlike standard beamforming in the conventional phased array ultrasound testing, randomly exciting a plurality of regions of the single probe can realize incoherent wavefront emission. Correspondingly, the echo signal is also modulated by the mask and received by the piezoelectric ceramic 5. Therefore, the received compressed signal is viewed as a linear combination of echo signals from point scatterers, thereby establishing the linear imaging model. The 3D scene includes N pixels in total, u is the actual tested signal, i.e., the received compressed signal, and v represents the reconstructed 4D ultrasound signal, i.e., the unknown real image. As shown in FIG. 5, the linear imaging model is given by: u=Hv+n, where u is an M-dimensional column vector, H is an M×N-dimensional block matrix determined by a medium of the target region, v is an N-dimensional column vector, and n is an M-dimensional column vector whose samples represent zero-mean additive white Gaussian noise (AWGN) with a noise variance of σₑ².

Because of the limitations in temporal bandwidth, as well as in mask thickness distribution, it is not guaranteed that echo signals of all regions are uncorrelated. In order to introduce more diversity among regions, S distinct sets of randomly excited echoes are selected, thereby obtaining additional measurements containing new information. The structure of the block matrix H is as follows:

H=[H₁,H₂,H₃ . . . H_{S}]^{T}

Hᵣ=[h_{r,1},h_{r,2} . . . h_{r,N}]
where Hᵣ is a matrix with a size of K×N, r∈{1, . . . , S}, representing a K-length pulse-echo response time sequence at a pixel n∈{1, . . . , N} in the image excited by different array elements in each random switching. Thus, M=KS, and u is also a column vector combination of S K-dimensional column vectors.

After the linear imaging model is established, image reconstruction requires inverting to solve for the vector v. Comparing the number of measurements M with the number of to-be-reconstructed pixels N, the linear system is underdetermined (M<N) at this time, with infinitely many possible solutions \hat{v} satisfying u=H\hat{v}+n. Herein, an optimal solution of the \hat{v} is searched by minimizing the cost function:

$${min}_{\hat{v}} {‖ H \hat{v} - u ‖}_{2}^{2} .$$

Matrix inversion can be implemented using the preconditioned conjugate gradient (PCG) method, and finally \hat{v}=H^{T}(HH^{T})⁻¹u can be obtained. To characterize accuracy of the image reconstruction, the peak signal-to-noise ratio (PSNR) is defined as:

$$PSNR = 10 {log}_{10} ( \frac{max ( v )}{M S E} )$$

The mean square error (MSE) is defined as:

$$M S E = \frac{1}{N} {\sum }_{n = 1}^{N} {( {v}_{n} - {\hat{v}}_{n} )}^{2}$$

where PSNR represents the peak signal-to-noise ratio, and the MSE represents the mean square error.

### Embodiment 1

First of all, a piezoelectric ceramic wafer with a frequency of 5 MHz is selected. Electrodes on one side of the piezoelectric ceramic wafer are segmented by a laser marking machine into 16×16 regions. Upon completion of segmentation, the electrodes are welded to an FPC board with 256 contacts. Meanwhile, a negative electrode on the other side of piezoelectric ceramic wafer is welded to a common cathode junction of the FPC board. Then, the backing is filled, and the piezoelectric ceramic wafer is assembled into the housing. The matching layer is spin-coated, with a thickness controlled to a quarter of the wavelength at this frequency in water, i.e., approximately 75 μm.

Further, a phase delay mask with 256 randomly distributed thicknesses (ranging between 0 and h) is fabricated using 3D printing. The sound velocity of the resin selected for the 3D printing is 2,350 m/s, while the sound velocity of the water is 1,450 m/s. When the thickness h is 0.85 mm, the phase delay of the incident wave can be modulated to π. The delay mask is then adhered to the surface of the probe with an ordinary water-based ultrasonic coupling agent.

Further, after the single-probe system with segmented electrodes is assembled, the probe is immersed in the water, and a hydrophone is used to acquire the wave field close to the mask. The transmitted ultrasound field at this time is recorded as p₀(x, y, z=0), and the ultrasound field on any plane is obtained with the angular spectrum method as P′(kₓ, k_{y}, z=1). Further, the pulse-echo signal is acquired by performing autoconvolution on the acquired hydrophone signal. 100 regions are randomly switched for multiple times, and all regions are used for acquisition. The block matrix H in the linear system is then filled.

As shown in FIG. 7, a simulated target region is set. The target region is scanned with the probe. Different regions are switched repeatedly for emission, and all regions are used for receiving the echoes. Solving the linear equation can complete the image reconstruction of the target region. As shown in FIG. 8, after the regions are switched repeatedly and acquired, the PSNR is 17.2123 dB, and the simulated defect can be clearly identified. By moving the probe to other regions in real time, the 3D dynamic imaging can be completed.

### Embodiment 2

First of all, a piezoelectric ceramic wafer with a frequency of 5 MHz is selected. Electrodes on one side of the piezoelectric ceramic wafer are segmented by a laser marking machine into 12×12 regions. Upon completion of segmentation, the electrodes are welded to an FPC board with 144 contacts. Meanwhile, a negative electrode on the other side of piezoelectric ceramic wafer is welded to a common cathode junction of the FPC board. Then, the piezoelectric ceramic wafer is assembled into the housing. The backing is filled. The matching layer is spin-coated, with a thickness controlled to a quarter of the wavelength at this frequency in water, i.e., approximately 75 μm.

Further, a phase delay mask having 144 different thicknesses (ranging from 0 to h) is machined on a flat plate with a computer numerical control (CNC) machining center. The sound velocity of the selected resin material is 2,350 m/s, while the sound velocity of the water is 1,450 m/s. When the thickness h is 0.85 mm, the phase delay of the incident wave can be modulated to T. The delay mask is then adhered to the surface of the probe with an ordinary water-based ultrasonic coupling agent.

Further, after the single-probe system with segmented electrodes is assembled, the probe is immersed in the water, and a hydrophone is used to acquire the wave field close to the mask. The transmitted ultrasound field at this time is recorded as p₀(x, y, z=0), and the ultrasound field on any plane is obtained with the angular spectrum method as P′(kₓ, k_{y}, z=1). Further, the pulse-echo signal is acquired by performing autoconvolution on the acquired hydrophone signal. 50 regions are randomly switched for multiple times, and all regions are used for acquisition. The block matrix H in the linear system is then filled.

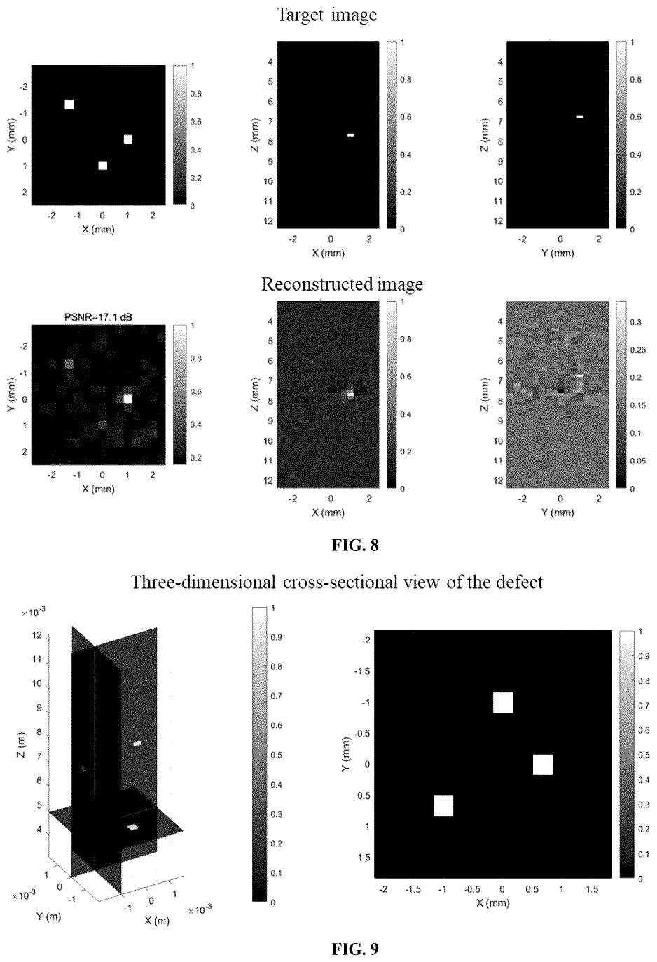

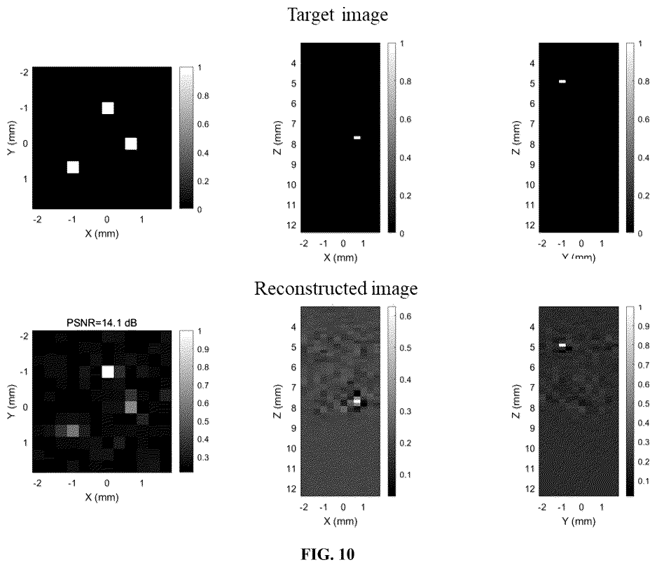

As shown in FIG. 9, a simulated target region is set. The target region is scanned with the probe. Different regions are switched repeatedly for emission, and all regions are used for receiving the echoes. Solving the linear equation can complete the image reconstruction of the target region. As shown in FIG. 10, after the regions are switched repeatedly and acquired, the PSNR is 14.1282 dB, and the simulated defect can be clearly identified. By moving the probe to other regions in real time, the 3D dynamic imaging can be completed.

The present disclosure has the following innovation points:
1) The ultrasound radiation surface of the piezoelectric ceramic 5 is segmented into L×L electrode regions. The control circuit is configured to control independent excitation and reception of each electrode region. Unlike phased array ultrasound transducers, the ultrasound radiation surface of the piezoelectric ceramic 5 and the control circuit thereof neither involve complex logic circuits, nor control the excitation time sequence of each array element through an electronic delay. The control circuit realizes multi-element cooperative control such as single-transmit-all-receive and multiple-transmit-all-receive on the electrode regions, so the structure is simpler than the existing 4D ultrasound imaging system.
2) At present, additional signal measurements are obtained by rotating or moving the mask. However, in the present disclosure, the spatial phase mask 7 is segmented into L×L mask regions with different thicknesses, and the mask regions are used with the electrode regions on the ultrasound radiation surface of the piezoelectric ceramic 5. By randomly exciting different regions each time to acquire multiple sets of “multiple-transmit single-receive” signals, the present disclosure effectively solves the prior art problem of difficulty in rotating or moving the mask to obtain additional signal measurements.
3) In the present disclosure, the linear system model reconstructs the 4D ultrasound image of the target region by: u=Hv+n. Compared with existing methods for reconstructing the 4D ultrasound image of the target region, the present disclosure is simple and can clearly identify the simulated defect.

The present disclosure and the embodiments thereof are described as above, and the description is not limiting; and what is shown in the drawing is only one of the embodiments of the present disclosure, and the actual structure is not limited thereto. All in all, if a person of ordinary skill in the art is inspired by the present disclosure, and designs, without departing from the purpose of the present disclosure, structural modes and embodiments similar to the technical solutions without inventive step, they shall fall within the scope of protection of the present disclosure.

1. A four-dimensional (4D) ultrasound imaging system based on electrode segmentation and compressive sensing, configured to acquire a 4D ultrasound image of a target region, and comprising: a housing, a cable, a backing, a piezoelectric ceramic, a matching layer, a spatial phase mask, and a control circuit, wherein
the backing, the piezoelectric ceramic, and the matching layer are sequentially disposed in the housing; a positive electrode surface of the piezoelectric ceramic serves as an ultrasound radiation surface and faces the target region; the backing is configured to absorb a reflected wave and is disposed on a negative electrode surface of the piezoelectric ceramic; the piezoelectric ceramic is electrically connected to the control circuit via the cable extending out of the housing; and the spatial phase mask is disposed outside the housing, comes into contact with a side of the matching layer away from the piezoelectric ceramic, and is configured to modulate a spatial phase distribution of a transmitted ultrasound wave; and
the ultrasound radiation surface of the piezoelectric ceramic is segmented into L×L electrode regions; each of the electrode regions is electrically connected to the control circuit; the control circuit is configured to control independent excitation and reception of each of the electrode region; and a negative electrode of the piezoelectric ceramic is grounded.

2. The 4D ultrasound imaging system based on electrode segmentation and compressive sensing according to claim 1, further comprising a linear compressive sensing module, wherein the linear compressive sensing module is electrically connected to the control circuit, and configured to reconstruct the 4D ultrasound image of the target region; and
a locking knob, wherein the locking knob is disposed on a top surface of the housing, and configured to fix the cable extending out of the housing.

3. The 4D ultrasound imaging system based on electrode segmentation and compressive sensing according to claim 1, wherein a size of the spatial phase mask is the same as a size of the piezoelectric ceramic; the spatial phase mask is segmented into L×L mask regions; the L×L mask regions have different thicknesses; a thickness relation between the L×L mask regions meets a condition that a spatial phase difference between transmitted waves of any two of the L×L mask regions in the spatial phase mask is greater than or equal to 0, and less than or equal to π; and the spatial phase difference between the transmitted waves of the any two of the L×L mask regions is set by:
φ=arg(P1−P2)
wherein φ represents the spatial phase difference between the transmitted waves, arg( ) represents a complex argument, and P1 and P2 are respectively complex representations of the transmitted waves of the any two of the L×L mask regions.

4. The 4D ultrasound imaging system based on electrode segmentation and compressive sensing according to claim 1, wherein both the backing and the matching layer are made of epoxy resin mixed with tungsten powder; a mass ratio of the tungsten powder to the epoxy resin in the backing is (2-4):1; a mass ratio of the tungsten powder to the epoxy resin in the matching layer is 1:(3-5); and a thickness of the matching layer is a quarter of a wavelength at a center frequency of the piezoelectric ceramic.

5. The 4D ultrasound imaging system based on electrode segmentation and compressive sensing according to claim 1, wherein the ultrasound radiation surface of the piezoelectric ceramic is segmented into the L×L electrode regions by any one of following methods: laser cutting, cutting by a dicing device, or sputtering deposition of a metal electrode layer on the piezoelectric ceramic.

6. The 4D ultrasound imaging system based on electrode segmentation and compressive sensing according to claim 1, wherein the ultrasound radiation surface of the piezoelectric ceramic is provided with a multi-electrode flexible printed circuit (FPC) board or pin electrodes.

7. A four-dimensional (4D) ultrasound imaging method applied to the 4D ultrasound imaging system based on electrode segmentation and compressive sensing according to claim 1, comprising following steps:
S1: performing wave field scanning on the target region to obtain a system matrix, and establishing a linear system model according to the system matrix;
S2: randomly exciting an electrode region of the piezoelectric ceramic to emit an ultrasound wave, wherein the ultrasound wave is modulated by the spatial phase mask and then reflected by the target region to obtain an echo signal;
S3: allowing the echo signal to pass through the spatial phase mask and to be received by the piezoelectric ceramic to form a compressed signal, and recording, by the control circuit, the compressed signal and transmitting the compressed signal to the linear system model; and
S4: repeating the step S2 and the step S3 sequentially multiple times, wherein the electrode regions of the piezoelectric ceramic are randomly controlled for excitation each time until the linear system model receives S compressed signals, and reconstructing, by the linear system model, the 4D ultrasound image of the target region according to the S compressed signals.

8. The 4D ultrasound imaging method according to claim 7, wherein the wave field scanning is performed by the following method:
acquiring, by using a hydrophone in a plane perpendicular to an ultrasound propagation direction, the ultrasound wave modulated by the spatial phase mask, processing the ultrasound wave modulated by the spatial phase mask to obtain wave field data, propagating, by using an angular spectrum method, the wave field data to planes at different propagation distances to obtain wave field data at the different propagation distances, and constructing the system matrix according to the wave field data at the different propagation distances.

9. The 4D ultrasound imaging method according to claim 7, wherein the linear system model reconstructs the 4D ultrasound image of the target region by:
u=Hv+n
wherein u represents the compressed signal, v represents a reconstructed 4D ultrasound image signal, n represents a noise signal, and H represents the system matrix.

10. The 4D ultrasound imaging method according to claim 7, wherein the 4D ultrasound imaging system further comprises a linear compressive sensing module, wherein the linear compressive sensing module is electrically connected to the control circuit, and configured to reconstruct the 4D ultrasound image of the target region; and
a locking knob, wherein the locking knob is disposed on a top surface of the housing, and configured to fix the cable extending out of the housing.

11. The 4D ultrasound imaging method according to claim 7, wherein a size of the spatial phase mask is the same as a size of the piezoelectric ceramic; the spatial phase mask is segmented into L×L mask regions; the L×L mask regions have different thicknesses; a thickness relation between the L×L mask regions meets a condition that a spatial phase difference between transmitted waves of any two of the L×L mask regions in the spatial phase mask is greater than or equal to 0, and less than or equal to π; and the spatial phase difference between the transmitted waves of the any two of the L×L mask regions is set by:
φ=arg(P1−P2)
wherein φ represents the spatial phase difference between the transmitted waves, arg( ) represents a complex argument, and P1 and P2 are respectively complex representations of the transmitted waves of the any two of the L×L mask regions.

12. The 4D ultrasound imaging method according to claim 7, wherein both the backing and the matching layer are made of epoxy resin mixed with tungsten powder; a mass ratio of the tungsten powder to the epoxy resin in the backing is (2-4):1; a mass ratio of the tungsten powder to the epoxy resin in the matching layer is 1:(3-5); and a thickness of the matching layer is a quarter of a wavelength at a center frequency of the piezoelectric ceramic.

13. The 4D ultrasound imaging method according to claim 7, wherein the ultrasound radiation surface of the piezoelectric ceramic is segmented into the L×L electrode regions by any one of following methods: laser cutting, cutting by a dicing device, or sputtering deposition of a metal electrode layer on the piezoelectric ceramic.

14. The 4D ultrasound imaging method according to claim 7, wherein the ultrasound radiation surface of the piezoelectric ceramic is provided with a multi-electrode flexible printed circuit (FPC) board or pin electrodes.
